(19) United States
(12) Patent Application Publication
(10) Pub. No.: US 2023/0095509 A1
(43) Pub. Date: Mar. 30, 2023

(54) NON-FIBRILLAR HEMOGLOBIN S OLIGOMERS AND METHODS TO IDENTIFY MODULATORS OF HEMOGLOBIN S POLYMERIZATION

(71) Applicant: REGENTS OF THE UNIVERSITY OF MINNESOTA, Minneapolis, MN (US)

(72) Inventors: David Kevin Wood, Minneapolis, MN (US); Jonathan N. Sachs, St. Paul, MN (US); Nagamani Vunnam, Minneapolis, MN (US); Chih Hung Lo, Singapore (SG)

(21) Appl. No.: 17/953,981

(22) Filed: Sep. 27, 2022

Related U.S. Application Data
(60) Provisional application No. 63/248,676, filed on Sep. 27, 2021.

(51) Int. Cl. G01N 33/72; G01N 21/64

(52) U.S. Cl. CPC G01N 33/721; G01N 21/6428; G01N 2800/22; G01N 2021/6439

(57) ABSTRACT

Provided herein are deoxygenated sickle hemoglobin (HbS), at concentrations far below the threshold for nucleation and rapid polymerization, that form small temporally stable assemblies of multiple α2β2 tetramers. Also provided are methods for making and detecting the small temporally stable assemblies and methods for identifying compounds that alter the structure of the small temporally stable assemblies.

### CROSS-REFERENCE TO RELATED APPLICATIONS

This application claims the benefit of U.S. Provisional Application Ser. No. 63/248,676, filed Sep. 27, 2022, which is incorporated by reference herein in its entirety.

### GOVERNMENT FUNDING

This invention was made with government support under HL 132906, R21HL152313, and R35GM131814, awarded by the National Institutes of Health. The government has certain rights in the invention.

### BACKGROUND

Sickle cell disease (SCD) is a devastating hereditary disorder that afflicts tens of millions worldwide, contributing to shortened lifespan, high infant mortality rates, and significantly reduced quality of life. Despite the severe health need, treatment options for SCD are limited to four FDA-approved therapies: voxelotor, crizanlizumab-tmca, L-glutamine, and hyroxyurea. Out of these four drugs, only voxelotor directly binds to hemoglobin and prevents sickle hemoglobin (HbS) polymerization by increasing hemoglobin's affinity for oxygen and stabilizing the oxy-hemoglobin state (Dufu et al., 2018, Clin Hemorheol Microcirc 70, 95-105; Oksenberg et al., 2016, Br J Haematol 175, 141-153). L-glutamine reduces oxidative damage to red blood cells by improving the redox potential of nicotinamide adenine dinucleotide (Jafri et al., 2022, Ann Hematol, 101(8):1645-1654), and Crizanlizumab-tmca binds to P-selectin and blocks interactions between endothelial cells, platelets, red blood cells, and leukocytes (Ataga et al., 2017, N Engl J Med 376, 429-439). Hydroxyurea upregulates fetal hemoglobin, which can inhibit HbS polymerization (Charache et al., 1995, N Engl J Med 332, 1317-1322 (1995); Cokic et al., 2003, J Clin Invest 111, 231-239 (2003)). Notably, two of these treatments were existing molecules for other indications and none of the four has been demonstrated clinically as a potent therapy that dramatically improves outcomes for most or all patients. While gene therapy has elicited considerable excitement, the need for bone marrow ablation and the high cost preclude broad application of this approach for the foreseeable future, while millions worldwide would benefit from new, affordable and easily accessible therapies.

### SUMMARY OF THE APPLICATION

The primary cause of sickle cell disease is the polymerization of sickle hemoglobin (HbS) in the deoxy condition. Over time, sickle cell disease can lead to complications such as infections, stroke, hypertension, episodes of pain, and organ damage. A blood and bone marrow transplant is currently the only cure for sickle cell disease, and these treatments are both costly and risky for patients. Therefore, there is desperate need to discover affordable small molecule-based therapies for sickle cell disease. Targeting polymerization of sickle hemoglobin is an attractive strategy but measuring polymerization in the context of drug discovery is difficult due to the high concentrations of HbS necessary and the rapid kinetics of the process. Thus, high-throughput screening (HTS) methods are largely non-existent. The inventors have developed methods that permit measurement of pre-fibrillar oligomers at concentrations of HbS much lower than those needed for polymerization, and this measurement can be measured at steady state, thus obviating complications due to kinetics. The methods can be used for discovering small molecules that directly inhibit HbS oligomerization and improve blood flow, potentially overcoming a long-standing bottleneck in sickle cell disease drug discovery.

Provided herein are methods for identifying compounds that alter intermolecular contact between two proteins. In one embodiment, the method includes providing a composition comprising low molecular weight sickle hemoglobin (HbS) assemblies, adding a test compound to the composition, and measuring a change in the structure of the low molecular weight HbS assemblies. Also provided is a low molecular weight HbS assembly that includes at least 2 HbS monomers, where the HbS monomer includes a tetramer. The tetramer typically includes two alpha subunits and two beta subunits, where the beta subunits each include the sickle cell point mutation. The present disclosure also provides a composition that includes the low molecular weight HbS assembly.

Terms used herein will be understood to take on their ordinary meaning in the relevant art unless specified otherwise. Several terms used herein and their meanings are set forth below.

As used herein, the term “protein” refers broadly to a polymer of two or more amino acids joined together by peptide bonds. The term “protein” also includes molecules which contain more than one protein joined by disulfide bonds, ionic bonds, or hydrophobic interactions, or complexes of polypeptides that are joined together, covalently or noncovalently, as multimers (e.g., dimers, trimers, tetramers). Thus, the terms peptide, oligopeptide, and polypeptide are all included within the definition of protein and these terms are used interchangeably. It should be understood that these terms do not connote a specific length of a polymer of amino acids, nor are they intended to imply or distinguish whether the protein is produced using recombinant techniques, chemical or enzymatic synthesis, or is naturally occurring.

As used herein, the term “HbS monomer” refers to a hemoglobin protein that is a tetramer of two alpha globin subunits and two beta globin subunits (an a2β2 tetramer), where the beta globin subunits include the mutation conferring the sickle-forming characteristic of red blood cells of the disease sickle-cell anemia.

As used herein, the term “low molecular weight HbS assemblies” and “low MW HbS assemblies” refers to the structures formed upon the incubation of a mixture of HbS monomers under conditions that result in less than 100% oxygen saturation of the HbS monomers, where the concentration of the HbS monomers is no greater than 1 millimolar (mM). A low molecular weight HbS assembly is temporally stable. The stability of this structure can be measured as described herein.

As used herein, the terms “FRET,” “fluorescence resonance energy transfer,” “Forster resonance energy transfer” and “resonance energy transfer” are used interchangeably, and refer to a nonradiative energy transfer process that occurs between two chromophores.

As used herein, a “chromophore” is a molecule that includes a region that adsorbs certain wavelengths of light and interacts with such a region of another chromophore so as to be useful for FRET. Chromophores suitable for use in a FRET assay are known to the skilled person and are readily available. In one embodiment, a chromophore may be a donor (also referred to as a donor probe). A donor probe refers to a molecule that will absorb energy and then re-emit at least a portion of the energy over time. In one embodiment, a chromophore may be an acceptor (also referred to as an acceptor probe). An acceptor probe refers to a molecule that will accept energy nonradiatively from a donor, thus decreasing the donor's emission intensity and excited-state lifetime. Thus, provided that a donor probe and an acceptor probe are physically located sufficiently close (most often within 2.5 to 12 nm), the two probes function together and, upon excitation with an appropriate wavelength, the donor probe transfers a precise amount of energy (proportional to the negative sixth power of the donor-acceptor distance) to the acceptor probe. This process can be specifically and quantitatively detected by observing the decrease in donor fluorescence intensity or lifetime or, in some cases, also the energy re-emitted by the acceptor probe as fluorescence. Thus, FRET assays are typically used to measure (1) the mole fraction of donors coupled with acceptor (e.g., to determine the binding affinity between the donor-labeled and acceptor-labeled molecules) and (2) the distance and/or distance changes between donor and acceptor. When donor and acceptor are both attached to the same molecule, FRET can be used to detect a change in the molecule's structure. When donor and acceptor are attached to different molecules, FRET can be used to detect a change in the relative positions (e.g., binding, orientation) and structures of the two molecules.

As used herein, the term “high-throughput screening” or “HTS” refers to a method drawing on different technologies and disciplines, for example, optics, chemistry, biology and/or image analysis, to permit rapid analysis of multiple samples at rates that permit highly parallel biological research and drug discovery. In some embodiments, HTS includes a step of detecting FRET in a sample, with the detection taking no longer than 10, no longer than 6, or no longer than 3 minutes to read all well of a 384-well or a 1536-well plate.

As used herein, “coefficient of variation” (CV) refers to a normalized measure of dispersion of a probability distribution or frequency distribution and is defined as the ratio of the standard deviation to the mean.

The term “and/or” means one or all of the listed elements or a combination of any two or more of the listed elements.

The words “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

The terms “comprises” and variations thereof do not have a limiting meaning where these terms appear in the description and claims.

It is understood that wherever embodiments are described herein with the language “include,” “includes,” or “including,” and the like, otherwise analogous embodiments described in terms of “consisting or and/or “consisting essentially or are also provided.

Unless otherwise specified, “a,” “an,” “the,” and “at least one” are used interchangeably and mean one or more than one.

Conditions that are “suitable” for an event to occur, such as expression of a tau protein, or “suitable” conditions are conditions that do not prevent such events from occurring. Thus, these conditions permit, enhance, facilitate, and/or are conducive to the event.

As used herein, “providing” in the context of a genetically engineered cell, a composition, an article, or a test compound means making the genetically engineered cell, composition, article, or test compound, purchasing the genetically engineered cell, composition, article, or test compound, or otherwise obtaining the genetically engineered cell, composition, article, or test compound.

Also herein, the recitations of numerical ranges by endpoints include all numbers subsumed within that range (e.g., 1 to 5 includes 1, 1.5, 2, 2.75, 3, 3.80, 4, 5, etc.).

Reference throughout this specification to “one embodiment,” “an embodiment,” “certain embodiments,” or “some embodiments,” etc., means that a particular feature, configuration, composition, or characteristic described in connection with the embodiment is included in at least one embodiment of the disclosure. Thus, the appearances of such phrases in various places throughout this specification are not necessarily referring to the same embodiment of the disclosure. Furthermore, the particular features, configurations, compositions, or characteristics may be combined in any suitable manner in one or more embodiments.

For any method disclosed herein that includes discrete steps, the steps may be conducted in any feasible order. And, as appropriate, any combination of two or more steps may be conducted simultaneously.

The above summary of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

The following detailed description of illustrative embodiments of the present disclosure may be best understood when read in conjunction with the following drawings.

FIG. 1A-1D show direct measurement of prefibrillar HbS oligomers by DLS and FRET. FIG. 1A shows Confirmation of oligomerization via DLS. Purified HbA or HbS (25 μM) was vacuum degassed with argon for 5 minutes and then DLS measurement were taken at room temperature. FIG. 1B, shows HbS oligomer size versus concentration. Purified HbS (0.01-100 μM) was degassed with argon for 5 minutes and then DLS measurement were taken at room temperature. FRET efficiency of fluorescent dyes, fluorescently labeled HbA and HbS samples under deoxygenated and oxygenated conditions. FRET efficiency of HbS sample was increased under deoxy condition, demonstrating that the signal is specific to association of HbS in the absence of fibers. Voxelotor and phenylalanine, which are known to inhibit polymerization, reduced FRET efficiency of HbS under deoxy conditions. FIG. 1C shows Schematic of TR-FRET scheme. Time-resolved FRET can be used to probe association of deoxy-HbS monomers in solution and to discover small molecules that disrupt these association. FIG. 1D shows FRET efficiency of fluorescent dyes, fluorescently labeled HbA and HbS samples under deoxygenated and oxygenated conditions. FRET efficiency of HbS sample was increased under deoxy condition, demonstrating that the signal is specific to association of HbS in the absence of fibers. Voxelotor and phenylalanine, which are known to inhibit polymerization, reduced FRET efficiency of HbS under deoxy conditions. All DLS data is reported as the effective diameter of the mean assembly size. Hypothesis testing was performed using parametric ANOVA with Dunnett's multiple comparison testing. *p-value<0.05; **p-value<0.01; ***p-value<0.001

FIG. 2A-2B show DLS characterization of HbS oligomers. FIG. 2A shows temporal stability of purified HbS under deoxy conditions. Purified HbS samples (25 μM) were incubated separately with hit compounds (100 μM) or DMSO only for two hours at room temperature and degassed with argon for five minutes, and then DLS measurement were taken at room temperature for two hours. (n=2) FIG. 2B Oligomer size of HbS and HbA (25 mM) measured by DLS in the presence or absence of oxygen and in the presence of DMSO.

FIG. 3A-3C shows lifetime FRET. FIG. 3A shows fluorescence lifetime measurements with HbS-rhodamine red (donor only) and HbS-rhodamine red HbS-Cy5 (FRET pair). The FRET efficiency of the HbS samples were calculated from lifetimes using Eq.1 FIG. 3 B shows histogram plots of all compounds from the LOPAC screen after removal of fluorescent compounds show an average FRET efficiency of 0.126±0.002. FIG. 3C shows fluorescence lifetime measurements with HbS-rhodamine red (donor only) in presence of DMSO, controls and three lead compounds.

FIG. 4A-4C shows lifetime FRET HTS. (FIG. 4A) Pilot screening with LOPAC library. Compounds that reduced the FRET efficiency below 3SD (black line) were selected for further characterization. Nine hit compounds with reproducible FRET change were identified from the pilot screens (hits below -3 SD). (B) Dose response curves of three HbS hit compounds (panel A, circled hits below -3 SD). Fluorescently labeled HbS samples were plated in a 1536 plate and treated separately with DMSO or the three hit compounds, chlormezanone, gabazine, and phosphoramidon disodium, in a dose-dependent manner from 0.1 to 1000 μM for 2 hours at room temperature and then vacuum degassed for 10 minutes, and then donor lifetimes were measured using a fluorescence plate reader. (FIG. 4C) Effective diameter of HbS in presence and absence of hit compounds from DLS measured at 90°. HbS samples (25 μM) were incubated separately with hit compounds (100 μM) or DMSO only for two hours at room temperature and degassed with argon for five minutes, and then DLS measurement were taken at room temperature. Hypothesis testing was performed using parametric ANOVA with Dunnett's multiple comparison testing. *p-value<0.05; **p-value<0.01; ***p-value<0.001 FIG. 5 shows Fibrillization Assay. Effect of hit compounds on polymerization of deoxyHbS in high potassium phosphate buffer. Curves represent growth of polymer as a function of time in the presence or absence of drug. Table shows slopes of lines fitted to linear part of the growth curve (approximate linear growth rate) for each condition as well as quality of fit.

FIG. 6a-6d shows chlormezanone binding confirmed by NMR. Superimposed 1D 1H NMR spectra showing chemical shift changes to HbS upon chlormezanone binding. The HbS (50 mM) only control is shown in black and HbS (50 mM) with chlormezanone (100 mM) is shown in red. Chemical shift perturbations observed upon binding chlormezanone in (FIG. 6a) Amide region; (FIG. 6b) aromatic and amide region region; (FIG. 6c) aliphatic region; and (FIG. 6d) methyl region. NMR signals attributed to free ligand are highlighted with asterisks. All spectra were collected on a Bruker Avance 900 MHz NMR Spectrometer at 288 K.

FIG. 7A-7B shows microfluidic device for measuring sickle blood flow. (FIG. 7A) Microfluidic device consisting of three polydimethylsiloxane (PDMS) layers: blood, hydration, and gas. High-speed cameras capture images to track blood flow in the region of interest (dashed box). (FIG. 7B) Transverse view of the multi-layered microfluidic device bonded to a glass slide.

FIG. 8A-C shows hit compounds reduce hypoxia-induced blood flow impairment. We treated RBC suspensions at 25% hematocrit from 7 individuals with SCD (genotype HbSS) with 500 μM of our hit compounds or voxelotor (positive control) and quantified the flow in our microfluidic platform under well-defined oxygen tensions. (FIG. 8A) Representative image of blood flowing in microfluidic device with overlay of arrows representing the velocities of features between frames using the Lucas Kanade Tomasi algorithm. (FIG. 8B) Area under velocity response curve (AUC) comparison among treated samples compared to the vehicle control. (FIG. 8C) Deceleration rate comparison among treated samples compared to the vehicle control. All statistics were performed using a one-tailed Wilcoxon signed rank test, a=0.05, * p-value<0.05, **p-value<0.01

FIG. 9A-9D shows quantification of sickle blood flow. (FIG. 9A) Raw oxygen and velocity recordings during microfluidic experiment. Arrow indicates the change in the steady state mean velocity after introducing hypoxic conditions on the microfluidic chip. (FIG. 9B) The on-chip oxygen concentration is cycled seven times during the experiment between 21% 02 and hypoxia. Changes in the velocity during each cycle are normalized to the average velocity of the initial 21% cycle. (FIG. 9C) Shaded region represents the area under the curve (AUC) using the steady state mean velocity changes at each oxygen concentration. This measurement quantifies the overall oxygen response of the blood by accounting for both the magnitude of the steady state mean velocity drop as well as the persistence of oxygen-dependence at higher oxygen concentrations (FIG. 9D) Blood deceleration is measured by a segmented moving mean of the velocity response during the hypoxic transition using Matlab signal processing. Inset shows moving mean (solid line) and linear curve fit (dashed) to determine the slope of the signal.

FIG. 10A-10C shows hit compounds do not increase hemoglobin oxygen binding affinity. Measurement of hemoglobin oxygen saturation in RBC from 4 healthy donors (genotype HbAA). RBC at 25% hematocrit were incubated with 500 uM voxelotor, chlormezanone, gabazine, or DMSO (control) for 1 hr at 37C and then diluted to 1% hematocrit with drug. Voxelotor significantly decreased the P50 of the treated RBC (p3.027). Chlormezanone and gabazine resulted in no significant (p>0.05) change in P50 relative to control.

### DETAILED DESCRIPTION OF ILLUSTRATIVE EMBODIMENTS

The inventors speculated that a high-throughput screening (HTS) strategy might be engineered to monitor whether small molecules can prevent the formation of pre-nucleation oligomers that form in dilute HbS solutions under hypoxic conditions. Pre-nucleating oligomers of deoxyHbS have been previously observed at concentrations relevant to nucleation and polymerization (˜1 mM) (Galkin et al., 2007, Biophysj 93, 902-913; Wang and Ferrone, 2013, Biophysj 105, 2149-2156), but as described herein these oligomers can be measured at orders of magnitude lower concentrations at room temperature. Specifically, the inventors made the unexpected determination that deoxygenated HbS, at concentrations far below the threshold for nucleation and rapid polymerization, forms small assemblies of multiple a2β2 tetramers that are temporally stable. This observation makes high-throughput screening (HTS) feasible. The methods described herein leverage high-sensitivity fluorescence measurements, including fluorescence lifetime measurements, that monitor these temporally stable, pre-fibrillar HbS oligomers. This approach is sensitive to compounds that inhibit HbS polymerization with or without modulating hemoglobin oxygen binding affinity. The methods were used in a pilot small-molecule screen describeed herein in which several novel inhibitors of HbS oligomerization were discovered. The novel inhibitors were validated in solution and in whole blood from sickle patients using ex vivo microfluidic measurements of blood flow.

### Proteins

Hemoglobin is a tetrameric protein that includes two pairs of two different subunits. The most common is hemoglobin A (also referred to as HbA), which has two alpha globin subunits and two beta globin subunits. Many variant forms of hemoglobin exist that cause disease, and one example is hemoglobin S or sickle hemoglobin. Sickle hemoglobin, like hemoglobin A, includes two alpha globin subunits and two beta globin subunits, but the beta globin subunits are encoded by a HBB gene having one specific point mutation that results in the substitution of glutamic acid for valine at the sixth amino acid of the beta globin subunit.

Sickle-cell disease (SCD), also known as sickle-cell anemia, is a blood disorder characterized by red blood cells that take on an abnormal and rigid sickle shape when the hemoglobin is deoxygenated. Sickling decreases flexibility of red blood cells and increases the risk of various complications. The process of sickling is driven by the intermolecular interaction of HbS monomers to assemble into a structure made up of multiple HbS monomers. As described herein, the inventors have identified conditions that allow the controlled formation of this structure, which is referred to herein as a low MW HbS assembly.

The present disclosure provides isolated low MW HbS assemblies and methods for making and using the low MW HbS assemblies.

The HbS monomers that make up a low MW assembly can be from a primate, such as a human. Examples of amino acid sequences of primate alpha globin subunits and beta globin subunits are known in the art. HbS monomers that can be used in the methods described herein are commercially available. Alternatively, useful HbS monomers can be isolated from blood, or the two subunits can be recombinantly produced in a host cell such as yeast and assembled into HbS monomers.

Whether a mixture of HbS tetramers form and maintain low MW HbS assemblies can be determined by measuring the size distribution profile of the tetramers in solution as a function of time. In one embodiment, the measurement is accomplished using dynamic light scattering (DLS); however, any method that measures the diameter of structures in the 5 nanometer (nm) to 50 nm range can be used. In one embodiment, low MW HbS assemblies can be prepared by incubating a mixture of HbS tetramers under conditions that reduce the amount of oxygen and result in the deoxygenation of at least some of the HbS monomers. In one embodiment, the concentration of HbS monomer used can be no greater than 1 mM, no greater than 500 micromolar (μM), no greater than 50 μM, or no greater than 25 μM. In one embodiment, the concentration of HbS monomer used can be at least 15 μM, at least 20 μM, or at least 25 μM. Examples of conditions that reduce the amount of oxygen include, but are not limited to, use of an oxygen scavenger, such as sodium metabisulfite (see Examples), or bubbling a non-oxygen containing gas through the mixture, such as nitrogen or argon. The mixture can be measured taking readings over time, such as readings every 30 seconds at 25° C. for two hours; however, other conditions can also be used to measure the presence and stability of pre-fibrillar. HbS monomers. A mixture of HbS monomers is considered to be a low MW HbS assembly if the mixture maintains, during a two hour assay at 25° C., a diameter within a range of no greater than 40 nm (the lowest reading and the highest reading differ by no greater than 40 nm), within a range of no greater than 35 nm, no greater than 30 nm, or no greater than 25 nm.

A HbS monomer optionally includes at least one chromophore. The chromophore can be a donor or an acceptor. Typically, a low MW HbS assembly includes two populations of HbS monomers. One population includes a first chromophore and the second population includes a second chromophore. Typically, the first and second chromophores are a donor-acceptor pair.

A chromophore can be covalently attached to a subunit of a HbS monomer. In one embodiment, a chromophore is covalently attached to a cysteine present on the subunit of a monomer. Alternatively, an affinity reagent such as biotin can be covalently attached to a subunit of a monomer, and a chromophore that includes an avidin can bind the chromophore to the subunit. The beta subunit includes a cysteine that is suitable for use in labeling the subunit with a chromophore.

Chromophores suitable for the methods described herein are known to the skilled person and are routinely used. Examples of suitable chromophores include, but are not limited to, fluorescent dyes, such as fluorescent dyes that can be attached to a protein when the protein is present in a cell. Examples of such dyes are known in the art and are routinely used. In those embodiments where two populations of monomers are present (where one population includes a chromophore and the second population includes a second chromophore), any appropriately selected two chromophores can be used as a donor-acceptor pair, provided that the energy emitted by a donor (the emission spectrum) overlaps with the energy absorbed by an acceptor (the excitation spectrum), e.g., an energy transfer process (FRET) occurs between two chromophores. A donor and an acceptor that meet this overlap are referred to as a donor-acceptor pair. In one embodiment, donor-acceptor pairs are chosen such that interference from test-compound fluorescence is minimized. Accordingly, in one embodiment, donors that can be excited at longer wavelengths are superior to those excitable at shorter wavelengths. For instance, donors that can be excited at wavelengths of greater than 300 nm, greater than 400 nm, or greater than 500 nm, are preferred in some embodiments. In some embodiment, red-shifted donors (greater than 600 nm, greater than 700 nm, or greater than 800 nm) can be used (Schaaf et al., 2018, Biosensors, 8(4):99). Also, probes with longer fluorescence lifetime (more than 3 nanoseconds (ns)) can be superior to probes with shorter fluorescence lifetime. Non-limiting examples of suitable fluorescent dyes include, but are not limited to, rhodamine red and Cy5. Examples of suitable donor-acceptor pairs include, but are not limited to, rhodamine red and Cy5.

Methods

The present disclosure provides methods of detection of a change to the intermolecular interaction between two HbS monomers. The methods include providing a composition that includes HbS monomers and adding a test compound to the composition. The composition includes conditions suitable for formation of, and/or maintaining the presence of, low molecular weight HbS assemblies. The conditions can include, for instance, low oxygen. Methods for reducing oxygen include, but are not limited to, the use of an oxygen scavenger, such as sodium metabisulfite. The HbS monomers of the composition can be present as low molecular weight HbS assemblies or the assembly formation can be triggered by altering the conditions to promote the formation of oligomers. In one embodiment the conditions include use of a mixture of HbS monomer at a concentration of at least 15, 25, or 75 micromolar (μM), and/or no greater than 100, 75, or 50 μM.

The method further includes adding a test compound to the composition and determining if there is a change in the structure of the low molecular weight HbS assemblies. A test compound that causes a change in the structure of the low molecular weight HbS assemblies is a potential lead compound that can be modified by medicinal chemistry and further tested in animal studies to identify potential therapeutics.

A compound useful in the method includes, but is not limited to, an organic compound, an inorganic compound, a metal, a polypeptide, a non-ribosomal polypeptide, a polyketide, or a peptidomimetic compound. The sources for compounds that may alter activity of a protein described herein include, but are not limited to, chemical compound libraries, fermentation media of Streptomycetes, other bacteria and fungi, and cell extracts of plants and other vegetations. Small molecule libraries are available, and include AMRI library, AnalytiCon, BioFocus DPI Library, Chem-X-Infinity, ChemBridge Library, ChemDiv Library, Enamine Library, The Greenpharma Natural Compound Library, Life Chemicals Library, LOPAC 1280TH, MicroSource Spectrum Collection, Pharmakon, The Prestwick Chemical Library®, SPECS, NIH Clinical Collection, Chiral Centers Diversity Library. In some embodiments, the number of compounds evaluated in an assay includes between 1 and 10,000,000 compounds, between 1 and 1,000,000 compounds, between 1 and 100,000 compounds, or between 1 and 1,000 test compounds.

In certain embodiments, the methods provided herein are carried out in a well of a plate with a plurality of wells, such as a multi-well plate or a multi-domain multi-well plate. Multi-well assay plates are useful in high throughput methods as they allow for the parallel processing and analysis of multiple samples distributed in multiple wells of a plate. Multi-well assay plates (also known as microplates or microtiter plates) can take a variety of forms, sizes and shapes (for instance, round- or flat-bottom multi-well plates). Examples of multi-well plate formats that can be used in the methods provided herein include those found on 96-well plates (12×8 array of wells), 384-well plates (24×16 array of wells), 1536-well plate (48×3 2 array of well), 3456-well plates and 9600-well plates. Other formats that may be used in the methods provided herein include, but are not limited to, single or multi-well plates comprising a plurality of domains. In certain embodiments, the plates are opaque-wall, opaque -bottom plates. In certain embodiments, the plates are black-wall, black-bottom plates. In certain embodiments, the plates have black walls and clear bottoms in order to allow bottom excitation and reading of fluorescence signals. In certain embodiments, the plates are chosen with minimal and uniform intrinsic fluorescence intensity within the range used in the method to avoid interference with FRET signals.

In one embodiment, dynamic light scattering (DLS) can be used detect a change to the intermolecular interaction between two HbS monomers. DLS measures the effective diameter of HbS present in a solution. The method includes use of conditions that result in the formation of low molecular weight HbS assemblies that are temporarily stable, and determining if the presence of a test compound results in a change in the effective diameter of the low molecular weight HbS assemblies. The change can be in increase or decrease in effective diameter. In one embodiment, the effective diameter of the low molecular weight HbS assemblies is reduced. In one embodiment the change is statistically significant. Without intending to be limiting, the change in diameter can be due to increased or reduced self-affinity of HbS monomers within low MW HbS assemblies, a change in the conformation of HbS monomers within low MW HbS assemblies, or a change in the arrangement of monomers within low MW HbS assemblies. DLS instruments useful in the methods disclosed herein are commercially available, including high throughput DLS instruments.

In another embodiment, FRET can be used detect a change to the intermolecular interaction between two HbS monomers. The first HbS monomer includes a donor chromophore, the second HbS monomer includes an acceptor chromophore, and the first and second chromophores are a donor-acceptor pair. Any method for detecting FRET signals, such as the decrease in donor fluorescence intensity or lifetime or, in some cases, also the energy re-emitted by the acceptor probe as fluorescence, can be used in the methods described herein.

In one embodiment, intensity-based FRET is used to detect changes in fluorescence. Methods for intensity-based FRET analysis are known to the person of skill in the art (Zeug et al., 2012, Biophys J. 103(9):1821-7. doi: 10.1016/j.bpj.2012.09.031). Instruments useful in the methods disclosed herein for intensity-based FRET analyses are commercially available, including high throughput intensity-based FRET instruments.

In another embodiment, the methods described herein use the lifetime of a chromophore instead of its intensity. A measuring instrument useful in the methods disclosed herein is a spectrometer that is compatible with FRET assays and can perform direct waveform recording to detect the entire time course of a time-resolved fluorescence decay with high quality (signal/noise >100) within 1 millisecond (ms) or less, in a microplate format that allows for the analysis of at least 100 samples per minute. An example of such an instrument is described by Petersen et al. (Rev Sci Instrum. 2014, 85(11):113101) and Schaaf et al. (SLAS Discov. 2017, 22(3): 250-261). An example of a laser suitable for the methods described herein is a passively Q-switched microchip laser (multi-wavelength series laser devices, model number FP2-473-3-10, manufactured by Concepts Research Corp., Charlotte, N.C., USA). An example of a digitizer suitable for the methods described herein is described in Pavicic (U.S. Pat. No. 6,816,102). An example of direct waveform recording suitable for the methods described herein is described in (Muretta, et al., 2010, Rev Sci Instrum 81:103101).

In one embodiment, FLT is measured using a format that permits rapid evaluation of multiple samples over a short period of time, e.g., a high-throughput format. In one embodiment, such a format is a plate reader (FLT-PR). FLT-PRs useful in the methods described herein are readily available (Fluorescence Innovations, Minneapolis, Minn.). The measurement of FLT by using direct waveform recording detection technology in a plate reader provides the precision to resolve small changes in FRET, and can scan the plate rapidly.

In one embodiment, a method includes identifying a compound that alters FRET of a low molecular weight HbS assembly. The alteration of FRET is evidence of a change in the distance between a donor chromophore of one monomer and an acceptor of another monomer, where both monomers are present in a low molecular weight HbS assembly. The skilled person will recognize that, within a mixture, some HbS monomers labeled with a donor chromophore and some HbS monomers labeled with an acceptor chromophore will disassociate and reform HbS monomers containing subunits having both donor and acceptor chromophores. Such hybrid HbS monomers occur at a low frequency due to the stability of HbS monomers and will not have a negative influence on the methods described herein.

The method includes providing a composition that includes two populations of HbS monomers and adding a test compound to the composition. The first HbS monomer includes a donor chromophore, the second HbS monomer includes an acceptor chromophore, and the first and second chromophores are a donor-acceptor pair.

The relative amount of donor chromophore to acceptor chromophore can be varied. For instance, the ratio of donor chromophore to acceptor chromophore can be 7:1, 6:1, 5:1, 4:1, 3:1: 1:1, 1:2, 1:3, 1:4, 1:5, 1:6, or 1:7.

The fluorescence lifetime of the donor probe, the acceptor probe, or a combination thereof is measured. A difference between the fluorescence lifetime in the presence of the test compound and the fluorescence lifetime in the absence of the test compound indicates that the test compound alters the FRET between the donor and acceptor probes. In one embodiment, the FRET between the donor and acceptor probes is reduced. An altered FRET indicates a change in the proximity of the two chromophores. For instance, a change in proximity can be the result of reduced intermolecular interaction between the monomers of an oligomer and disruption of the oligomers.

Measuring the fluorescence lifetime of a donor probe, an acceptor probe, or both, may occur over a specific time period. In one embodiment, the time period of measuring the fluorescence lifetime of a mixture is no greater than 5 seconds, no greater than 1 second, no greater than 0.5 seconds, no greater than 0.1 seconds, no greater than 0.01 seconds, no greater than 0.001 seconds, no greater than 0.0001 seconds, no greater than 0.00001 seconds, or no greater than 0.000005 seconds. This time period for measurement is distinct from the time period a donor probe fluoresces (i.e., the fluorescence lifetime of a donor probe), which is on the order of nanoseconds.

In one embodiment, the coefficient of variation (CV) obtained from a composition is no greater than 1%, no greater than 0.5%, or no greater than 0.3%.

In one embodiment, a waveform obtained from a composition has a signal/noise (S/N) that is at least 100, at least 200, at least 300, or at least 400.

In certain embodiments, the FRET assays disclosed herein are measured at a single emission wavelength. In certain embodiments, the FRET fluorescence lifetime properties are measured at two or more wavelengths.

Kits

Also provided herein are kits for identifying a compound that alters the interaction of HbS monomers. In one embodiment, a kit includes HbS monomer that includes a donor chromophore and HbS monomer that includes an acceptor chromophore, wherein the chromophores are a donor-acceptor pair. In another embodiment, a kit includes HbS monomer, donor chromophore, and acceptor chromophore, wherein the chromophores are a donor-acceptor pair.

In certain embodiments, a kit may further include buffers and reagents useful for the procedure, and instructions for carrying out the assay. In certain embodiments, a kit may further include other useful agents, such as positive and negative control reagents, and the like.

Methods and kits disclosed herein may be carried out in numerous formats known in the art. In certain embodiments, the methods provided herein are carried out using solid-phase assay formats. In certain embodiments, the methods provided herein are carried out in a well of a plate with a plurality of wells, such as a multi-well plate or a multi-domain multi-well plate. The use of multi-well assay plates allows for the parallel processing and analysis of multiple samples distributed in multiple wells of a plate. Multi-well assay plates (also known as microplates or microtiter plates) can take a variety of forms, sizes and shapes (for instance, round- or flat-bottom multi-well plates). Examples of multi-well plate formats that can be used in the methods provided herein include those found on 96-well plates (12×8 array of wells), 384-well plates (24 x 16 array of wells), 1536-well plate (48×3 2 array of well), 3456- well plates and 9600-well plates. Other formats that may be used in the methods provided herein include, but are not limited to, single or multi-well plates comprising a plurality of domains. In certain embodiments, the plates are opaque-wall, opaque -bottom plates. In certain embodiments, the plates are black-wall, black-bottom plates. In certain embodiments, the plates have black walls and clear bottoms in order to allow bottom excitation and reading of the fluorescence signals. In certain embodiments, the plates are chosen with minimal and uniform intrinsic fluorescence intensity within the range used in the method to avoid interference with the FRET signals.

The invention is defined in the claims. However, below there is provided a non-exhaustive listing of non-limiting exemplary aspects. Any one or more of the features of these aspects may be combined with any one or more features of another example, embodiment, or aspect described herein.

### Exemplary Aspects

Aspect 1 is a method for identifying a compound that alters intermolecular contact between two proteins, the method comprising: providing a composition comprising low molecular weight sickle hemoglobin (HbS) assemblies; adding a test compound to the composition; and measuring a change in the structure of the low molecular weight HbS assemblies.

Aspect 2 is the method of Aspect 1, wherein the low molecular weight HbS assemblies comprise a first sickle hemoglobin (HbS) tetramer and a second HbS tetramer, wherein the first HbS tetramer comprises a donor chromophore, wherein the second HbS tetramer comprises an acceptor chromophore, wherein the first and second HbS tetramers are deoxygenated, wherein the first and second HbS tetramers form a low molecular weight HbS assembly, and wherein the measuring comprises measuring fluorescence resonance energy transfer (FRET) of the donor chromophore, the acceptor chromophore, or the combination thereof.

Aspect 3 is the method of Aspect 1 or 2, wherein a difference between the FRET in the presence of the test compound and the FRET in the absence of the test compound is not detected.

Aspect 4 is the method of any one of Aspects 1-3, wherein a difference between the FRET in the presence of the test compound and the FRET in the absence of the test compound is detected.

Aspect 5 is the method of any one of Aspects 1-4, wherein the difference indicates that the test compound alters the FRET of the donor chromophore, the acceptor chromophore, or the combination thereof.

Aspect 6 is the method of any one of Aspects 1-5, wherein the ratio of donor to acceptor is 7:1,6:1,5:1,4:1,3:1: 1:1, 1:2, 1:3, 1:4, 1:5, 1:6,or1:7.

Aspect 7 is the method of any one of Aspects 1-6, wherein the providing comprises incubation of the composition at a concentration of 25 μM in the presence of an oxygen scavenger.

Aspect 8 is the method of any one of Aspects 1-7, wherein the FRET of the donor probe is changed in the presence of the test compound.

Aspect 9 is the method of any one of Aspects 1-8, wherein the FRET of the donor probe is reduced in the presence of the test compound.

Aspect 10 is the method of any one of Aspects 1-9, wherein the FRET of the donor probe is unchanged in the presence of the test compound.

Aspect 11 is the method of any one of Aspects 1-10, wherein the measuring comprises high-throughput screening.

Aspect 12 is the method of any one of Aspects 1-11, wherein the measuring comprises measuring dynamic light scattering (DLS) of the low molecular weight HbS assemblies.

Aspect 13 is the method of any one of Aspects 1-12, wherein a difference between the DLS in the presence of the test compound and the DLS in the absence of the test compound is not detected.

Aspect 14 is the method of any one of Aspects 1-13, wherein a difference between the DLS in the presence of the test compound and the DLS in the absence of the test compound is detected.

Aspect 15 is the method of any one of Aspects 1-14, wherein the difference indicates that the test compound alters the effective diameter of the low molecular weight HbS assemblies.

Aspect 16 is a low molecular weight HbS assembly comprising at least 2 HbS monomers, wherein the HbS monomer comprises a tetramer, the tetramer comprising two alpha subunits and two beta subunits, wherein the beta subunits each comprise the sickle cell point mutation.

Aspect 17 is the low molecular weight HbS assembly of any one of Aspects 1-16, wherein one or both beta subunits comprise a chromophore.

Aspect 18 is the low molecular weight HbS assembly of any one of Aspects 1-17, wherein the stable non-fibrillar oligomer comprises two populations of HbS monomers, wherein the first population comprises a donor chromophore and the second population comprises an acceptor chromophore.

Aspect 19 is a composition comprising the low molecular weight HbS assembly of any one of Aspects 1-18.

Aspect 20 is the composition of any one of Aspects 1-19, wherein the concentration of HbS monomers is at least 15 μM and no greater than 1 mM.

Aspect 21 is the composition of any one of Aspects 1-20, wherein the concentration of HbS monomers is no greater than 500 μM.

### EXAMPLES

The present invention is illustrated by the following examples. It is to be understood that the particular examples, materials, amounts, and procedures are to be interpreted broadly in accordance with the scope and spirit of the invention as set forth herein.

### Example 1

Fluorescence lifetime measurement of prefibrillar sickle hemoglobin oligomers as a platform for drug discovery in sickle cell disease

All the most promising therapeutic approaches for SCD proposed to date—gene therapy, fetal hemoglobin (HbF) induction, and hemoglobin oxygen affinity modulation—act by reducing the amount of polymer in sickle RBCs. However, the only FDA-approved drug targeting sickle hemoglobin (HbS) polymerization, voxelotor, has not yet shown broad efficacy and questions remain about its pharmacodynamics and long-term effects (Han et al., 2020, Pharmacotherapy 40, 525-534; Henry et al., 2021, Biophys J 120, 2543-2551; Henry et al., 2021, Blood 138, 1172 1181; Hebbel and Hedlund, 2018, Am J Hematol 93, 321-325). Moreover, only a handful of small molecule therapies have ever been proposed to target HbS polymerization directly. A major reason for this dearth of therapies is the lack of high-throughput screening (HTS) assays with which to identify molecules that can inhibit HbS polymerization. One challenge in developing HTS-compatible assays for HbS polymerization is that nucleation of polymer may begin in a wide temporal window, and the polymer growth is extremely rapid upon nucleation (Aprelev et al., 2011, Biophysj 101, 885-891; Coletta et al., 1982, NATURE-LONDON- 300, 194-197; Ferrone et al., 1985, J. Mol. Biol. 183, 611-631; Ferrone et al., 1985, J. Mol. Biol. 183, 591-610; Ferrone et al., 1980, Biophysj 32, 361-380; Castle et al., 2019, Sci Adv 5, eaau1086). Thus, measuring polymer growth kinetics in parallel conditions in 96-, 384-, or 1536-well plates is virtually impossible. The result is that, to our knowledge, no compounds that target HbS polymerization have been developed through traditional screening approaches that are the workhorse of pharmaceutical development.

The inventors hypothesized that it might be possible to engineer an HTS strategy to monitor whether small molecules can prevent the formation of pre-nucleation oligomers that form in dilute HbS solutions under hypoxic conditions. Pre-nucleating oligomers of deoxyHbS have been previously observed at concentrations relevant to nucleation and polymerization (˜1 mM) (Galkin et al., 2007, Biophysj 93, 902-913; Wang and Ferrone, 2013, Biophysj 105, 2149 2156), but here it is shown that these oligomers can be measured at orders of magnitude lower concentrations at room temperature, making HTS feasible. Based on this novel observation, the inventors developed an HTS-compatible assay using high-sensitivity lifetime FRET, and demonstrated that this assay is sensitive to compounds that inhibit deoxyHbS polymerization. Also reported here is the results of a pilot small-molecule screen in which several novel inhibitors of HbS oligomerization were discovered. Finally, the validation of those novel inhibitors in solution and in whole blood from sickle patients using ex vivo microfluidic measurements of blood flow is reported.

### Results

Pre-fibrillar HbS oligomers can be measured at sub-nucleating concentrations.

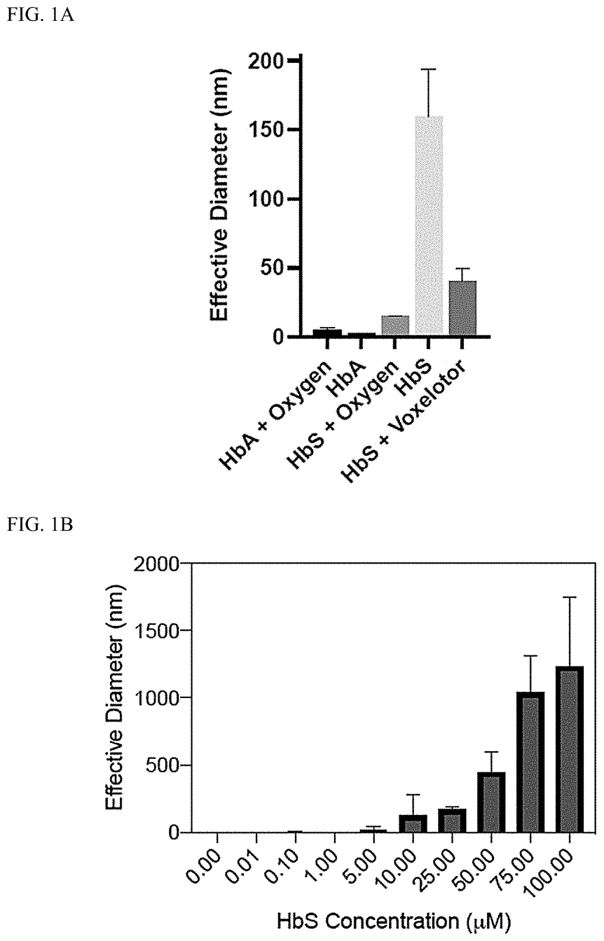

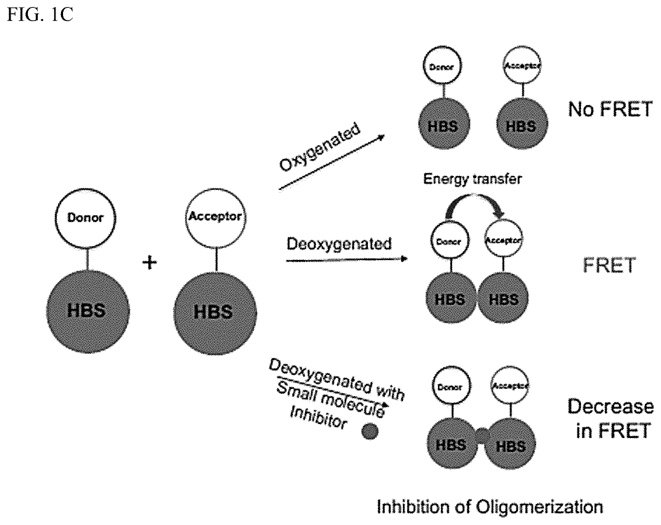

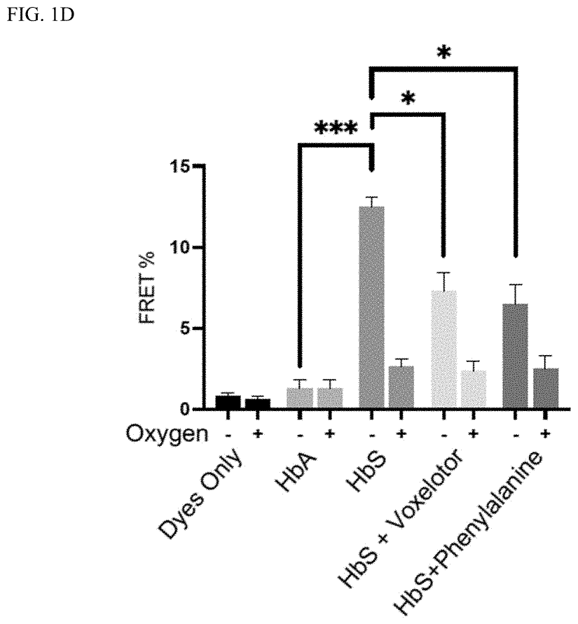

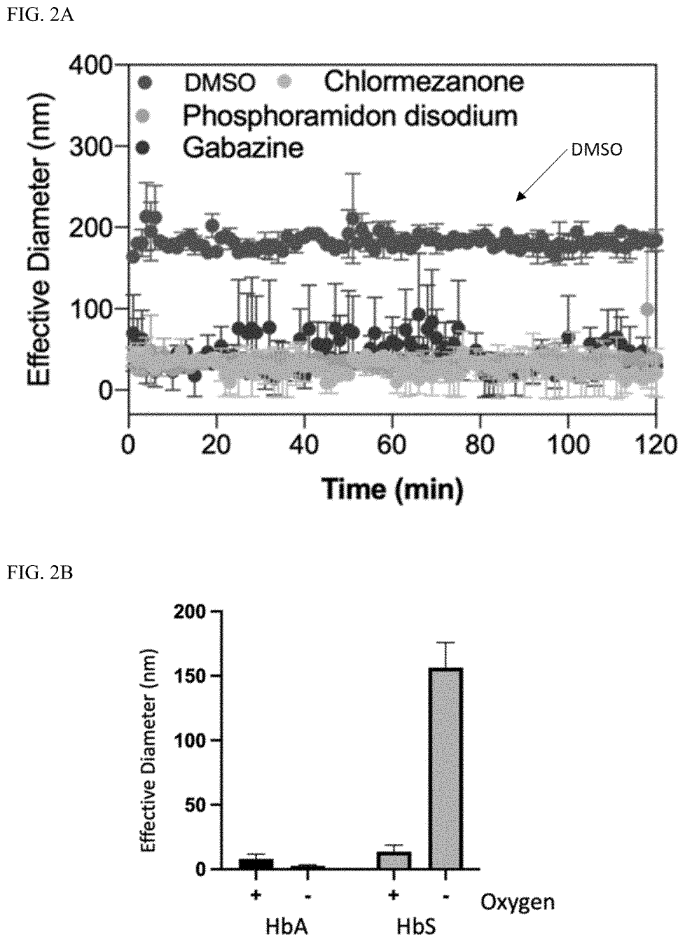

To test the hypothesis that deoxyHbS forms stable oligomers at sub-nucleating concentrations, we measured HbS solutions by dynamic light scattering (DLS) and found that HbS (but not HbA) formed oligomers larger than the tetrameric protein under fully anoxic conditions (FIG. 1A). We also found that deoxyHbS forms oligomers that vary in size with concentration (FIG. 1B). To test the hypothesis that these oligomers are sensitive to drugs that inhibit polymer formation, we measured them in the presence of voxelotor (a compound that increases hemoglobin oxygen binding affinity), which dramatically reduced the mean oligomer size (FIG. 1A). We also found that the oligomers were temporally stable (FIG. 2A) and that DMSO did not affect the oligomer size (FIG. 2B), two points that make HTS feasible.

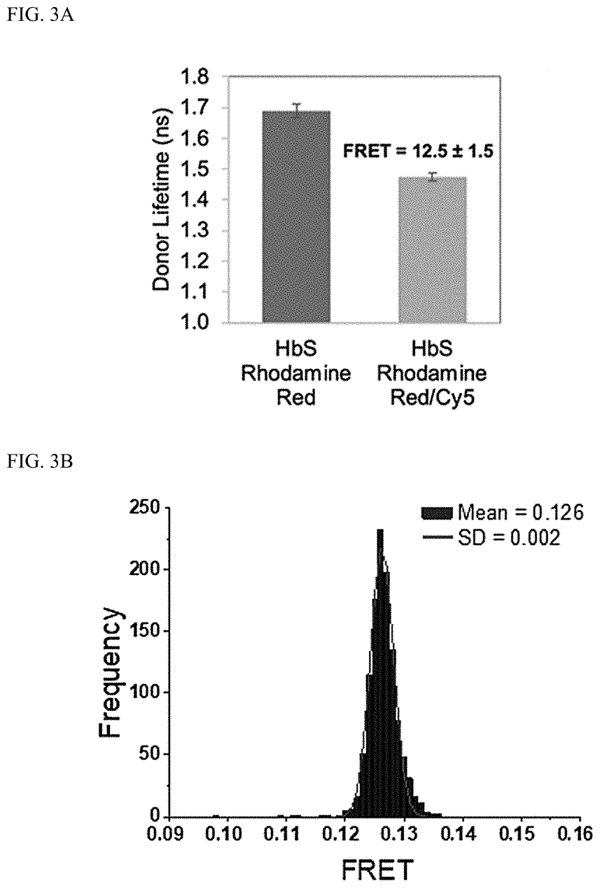

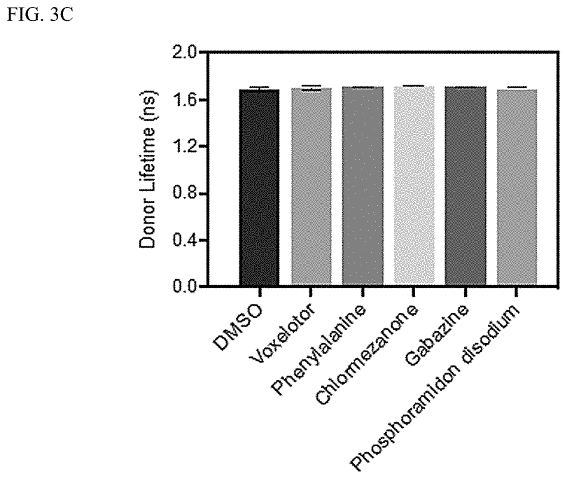

As an orthogonal modality to DLS, we developed a fluorescence lifetime (FLT) FRET assay (FIG. 1C) to measure deoxyHbS oligomers. As shown in FIG. 1D, measurements of deoxyHbS showed a substantial increase in FRET efficiency (measured as a decrease in the donor FLT in the presence of the acceptor compared with the donor only, FIG. 3A), which corresponds to association of the deoxyHbS molecules. By constrast, oxy- and deoxyHbA and oxyHbS showed no significant increase in FRET efficiency in the presence of the acceptor. We tested the effect of voxelotor and phenylalanine on the oligomerization of deoxyHbS (Oksenberg et al., 2016, Br J Haematol 175, 141-153; Dufu et al., 2018, Hematol Rep 10, 7419; Metcalf et al., 2017, ACS Med Chem Lett 8, 321-326), and both molecules decreased the FRET efficiency (FIG. 1D), demonstrating that the FRET-based assay is sensitive to compounds that are known to inhibit deoxyHbS polymerization.

High-throughput FLT FRET reveals novel inhibitors of HbS oligomerization.

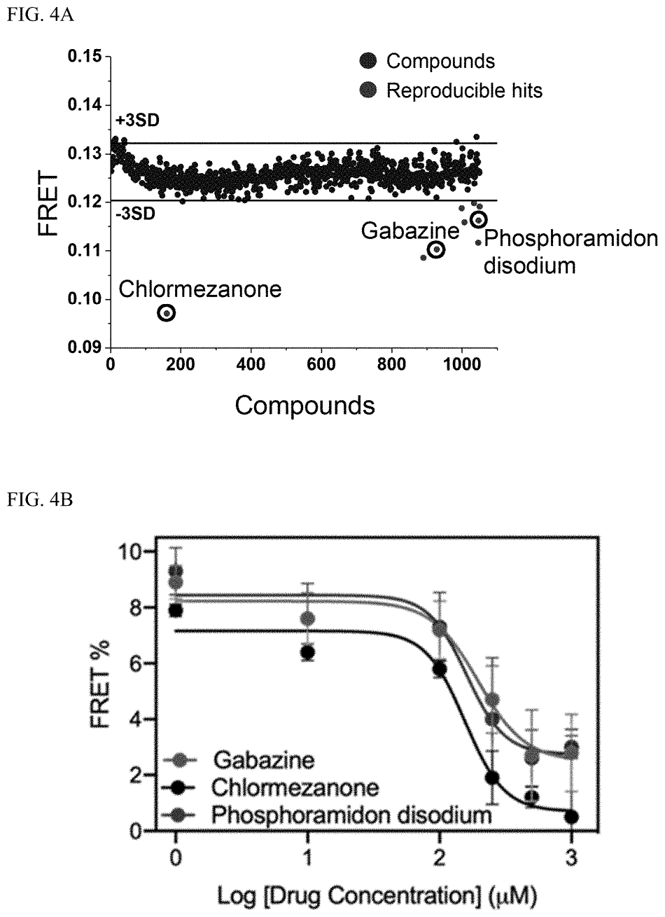

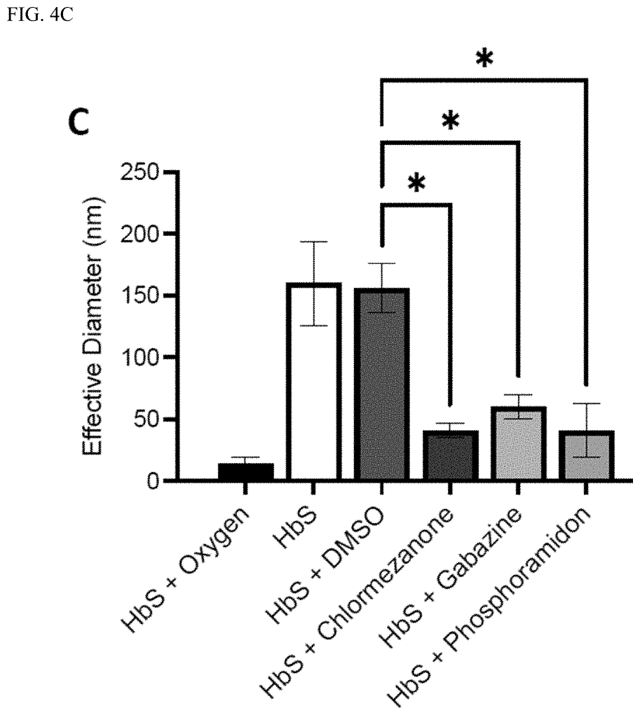

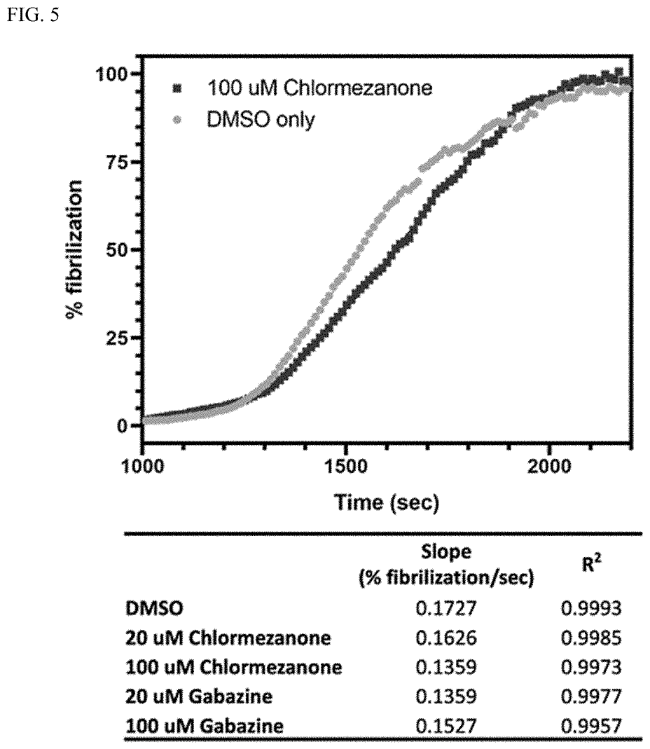

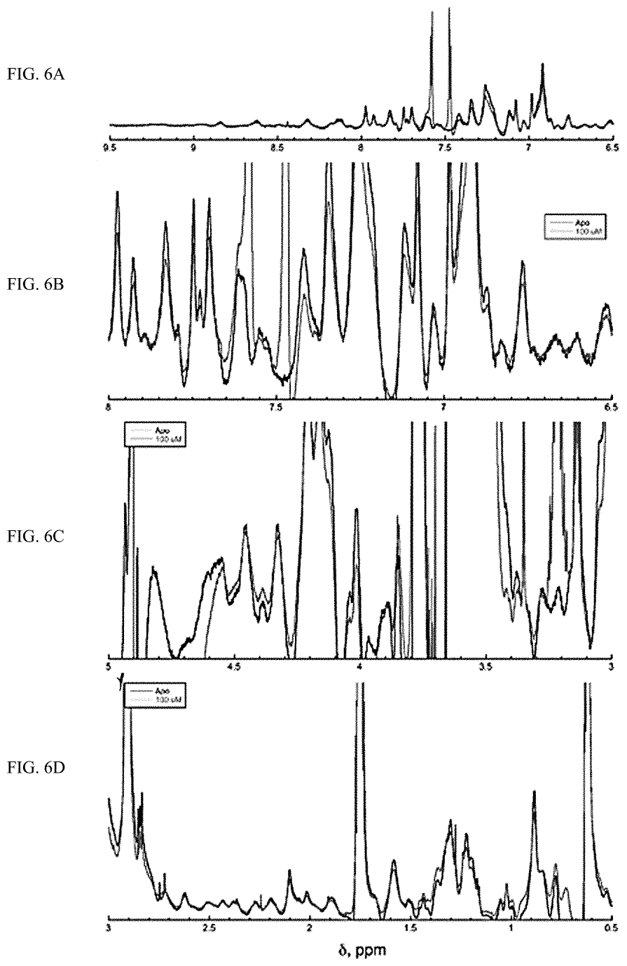

Using the lifetime FRET assay, we performed HTS of the LOPAC library (1284 compounds) as a pilot to determine if the method can identify small molecules that inhibit deoxyHbS oligomerization. The screen was enabled by a high-throughput FLT plate reader technology (Stroik et al., 2018, Sci Rep 8, 12560). FRET efficiencies from all compounds (that were not fluorescent) were averaged from three independent screens (FIG. 4A). We identified nine compounds that were at least 3a from the mean of all compounds, based on a Gaussian fit of the histograms (FIG. 3B). Four of these hit compounds reduced the FRET efficiency by more than 20% (FIG. 4A, circles below -3 SD), with chlormezanone having by far the largest effect. We also checked if controls and hit compounds acted as a quencher (thereby artificially decreasing the FRET signal without preventing oligomerization) by testing their effect on donor only lifetime. If compounds are fluorescence quenchers, we would see a change in donor only lifetime when compared with DMSO control. We did not observe a significant change is donor only lifetimes in presence of compounds (FIG. 3C). These results further confirm that decrease in FRET is due to inhibition of deoxyHbS oligomerization. A subset of hit compounds (chlormezanone, gabazine and phosphoramidon disodium) were evaluated using dose response with FRET (FIG. 4B), and all three compounds decreased FRET efficiency in a dose-dependent manner, again with chlormezanone showing the biggest effect at an IC50 >100 μM. We also measured the size of deoxyHbs oligomers using DLS in the presence and absence of hit compounds (FIG. 4C) and found that all three compounds significantly decreased the oligomer size relative to control (and to the same extent as voxelotor). We tested the ability of chlormezanone and gabazine to inhibit polymerization and found a modest, dose-responsive decrease in the rate of fibril growth with chlormezanone (20% at 100 μM, FIG. 5), with a less compelling result for gabazine. Unlike voxelotor (Oksenberg et al., 2016, Br J Haematol 175, 141-153), these initial hit compounds do not eliminate fibril growth altogether. Finally, we used NMR to directly confirm binding of chlormezanone to HbS (FIG. 6). Together these data suggest that our FLT screening method can reveal small molecules that bind to HbS and inhibit deoxyHbS oligomerization in solution, but that larger library screens may be needed to identify compounds that potently inhibit polymerization at physiological HbS concentrations.

Hit compounds reduce oxygen-dependent blood flow impairment.

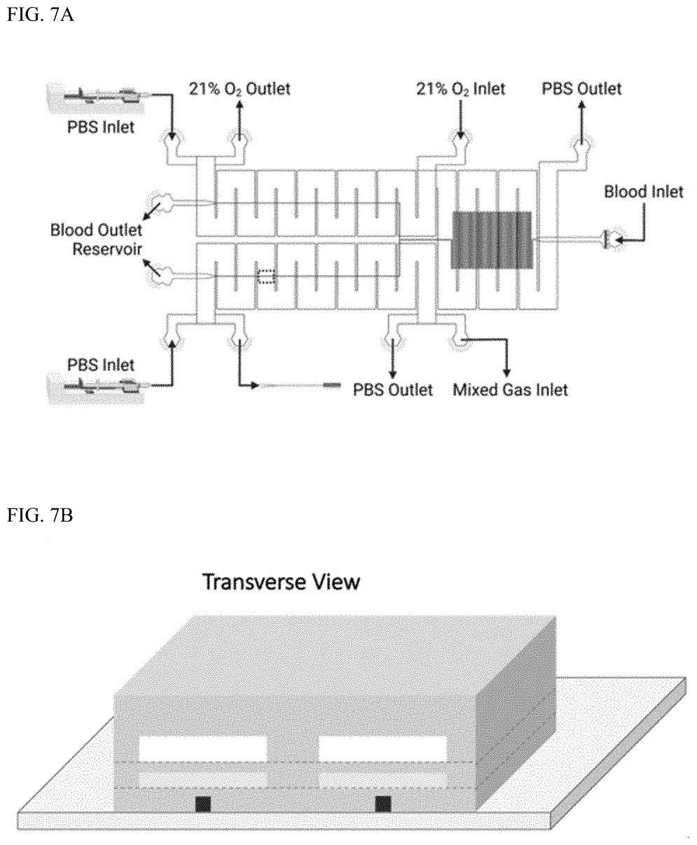

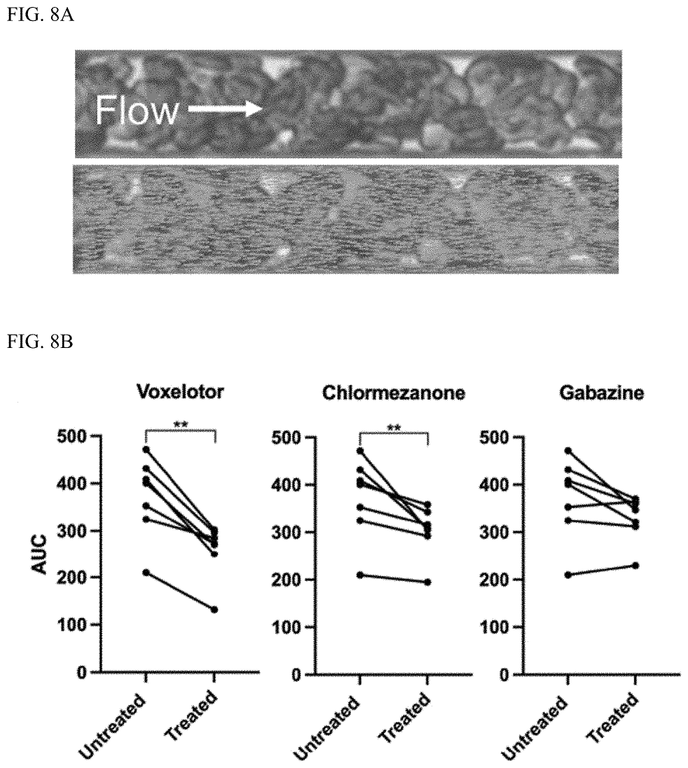

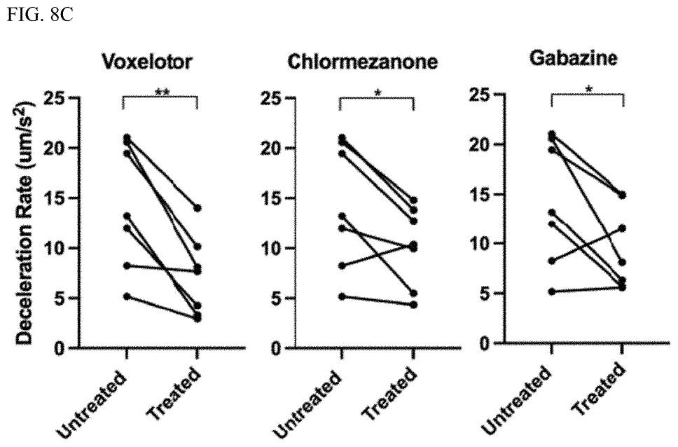

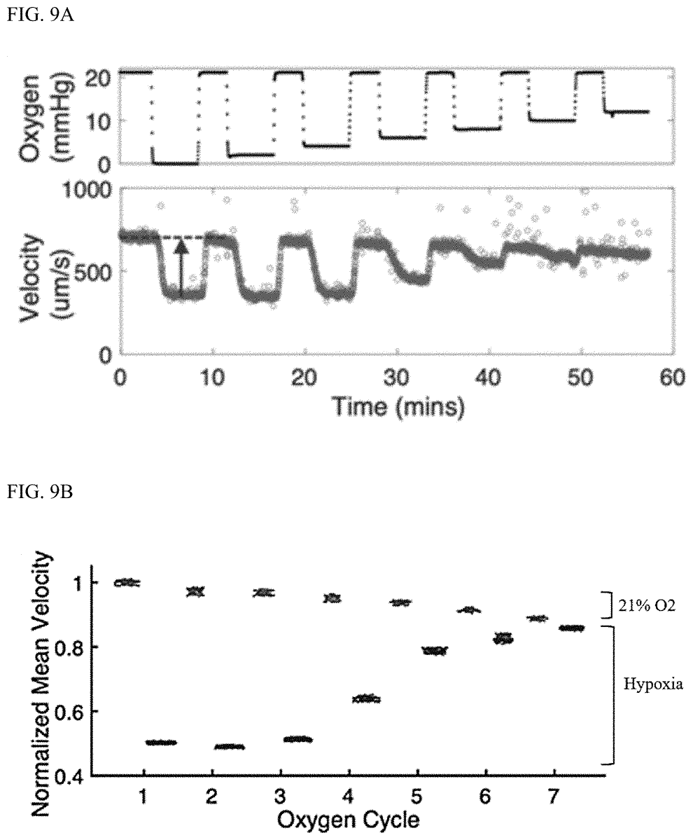

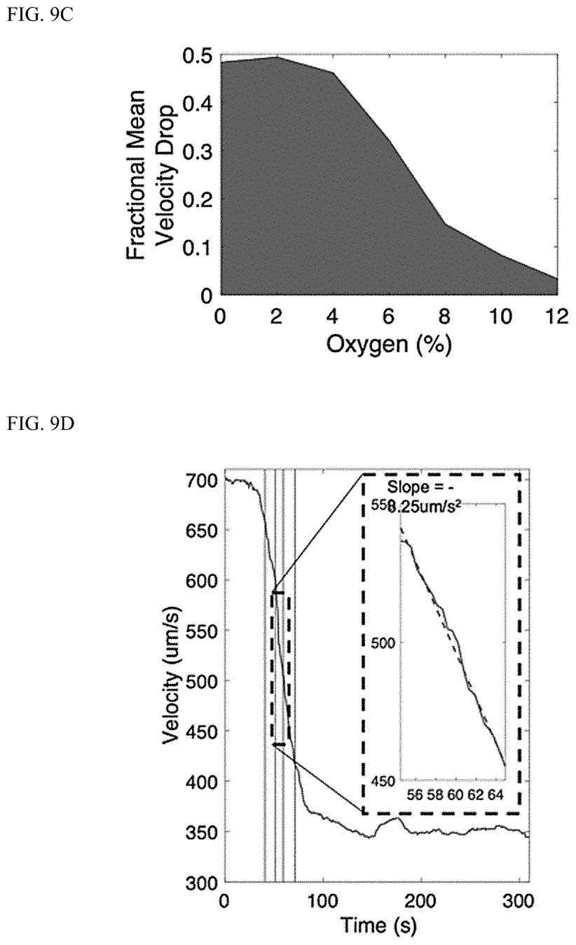

We evaluated whether the hits could reduce hypoxia-induced impairment of sickle blood flow using previously published microfluidic devices (FIG. 7, FIG. 8A) (Valdez et al., 2019, APL Bioengineering 3, 046101; Szafraniec et al.,2022, Lab Chip 22, 1565-1575. We treated RBC from 7 individuals with SCD (genotype HbSS) with 500 μM of chlormezanone, gabazine, or voxelotor (positive control) and quantified the oxygen-dependent flow in our microfluidic platform (FIG. 9A-B). The 500 μM dose represents the maximum whole blood concentration observed for the FDA-approved dosage of voxelotor and corresponds to 35-45% modification of the hemoglobin at 25% hematocrit (assuming complete RBC partitioning and 30-36 g/dl intracellular hemoglobin concentration) (Hutchaleelaha et al., 2019, Br J Clin Pharmacol 85, 1290-1302). We quantified the area under the velocity response curve (AUC, FIG. 9C) and the rate of deceleration (FIG. 9D) for each treatment condition. As shown in FIG. 3B, chlormezanone and voxelotor significantly reduced the mean AUC (p=0.0078), relative to control, while samples treated with gabazine showed an insignificant trend towards reduced AUC (p=0.0547). Chlormezanone, gabazine, and voxelotor all significantly reduced the deceleration rate of blood during deoxygenation (FIG. 8C, p=0.0391 for chlormezanone and gabazine, p=0.0078 for voxelotor).

Hit compounds do not increase hemoglobin oxygen affinity.

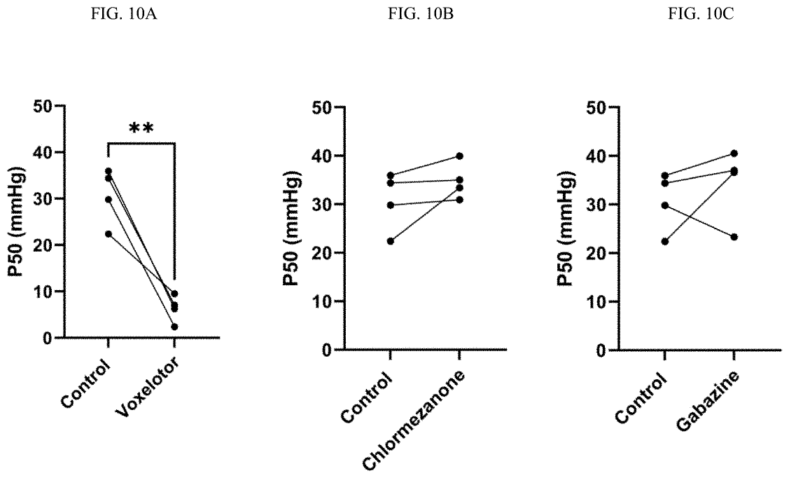

A mechanistic question is whether compounds discovered with our screening method inhibit oligomerization by increasing hemoglobin oxygen binding affinity, similar to voxelotor, or by some other mechanism. Using a previously described microfluidic platform (Di Caprio et al., 2019, Proc Natl Acad Sci USA 116, 25236-25242; Di Caprio et al., 2015, Proc Natl Acad Sci USA 112, 9984-9989), we quantified the oxygen binding curves of RBC from 4 healthy donors (genotype HbAA) with and without incubation with chlormezanone, gabazine, or voxelotor (positive control) at 500 μM. As expected, voxelotor significantly (p<0.05) decreases P50, indicating an increase in oxygen binding affinity (FIG. 10A). However, neither chlormezanone nor gabazine significantly shifted P50 (FIG. 10B-C), and for most of the samples tested, the trend was that these compounds slightly increased P50, strongly suggesting that they do not act by increasing oxygen binding affinity.

### DISCUSSION

The HTS strategy presented here, which uses high-sensitivity lifetime FRET (FIGS. 1C-D) to monitor temporally stable prefibrillar deoxyHbS oligomers (confirmed with DLS, FIG. 2B) circumvents the challenges of highly parallelized quantification of deoxyHbS polymerization such as high protein concentration and stochastic nucleation events. The use of FLT detection is a advantage of our assay because it increases the precision of FRET-based screening by a factor of 30 compared with conventional fluorescence intensity detection (Stroik et al., 2018, Sci Rep 8, 12560). Additionally, our labeling methodology overcomes the technical challenge that traditional FRET probes' emission peaks overlap with hemoglobin absorption peaks. The result is an excellent HTS assay with Z′β0.65 and the possibility of further optimization (Zhang et al., 1999, J Biomol Screen 4, 67-73). While we do not know the structure of the deoxyHbS oligomers measured in these studies, the sensitivity of the oligomers to phenylalanine and voxelotor strongly suggests that the oligomers share at least some critical bonds with the polymer, making them a suitable target for drug discovery. Moreover, because these are pre-nucleation oligomers, drugs that disrupt them are likely to inhibit or delay nucleation, which is the goal of most anti-sickling therapies (Eaton et al. 2017, Blood 129, 2719 2726).

We have shown that this breakthrough HTS assay is sensitive to compounds that inhibit deoxyHbS polymerization by increasing oxygen binding affinity (voxelotor, FIG. 1D). With voxelotor clinically approved and a new oxygen affinity modulator in clinical trial (Gopalsamy et al., 2021, J Med Chem 64, 326-342; Knee et al., 2021, Am J Hematol 96, E272-E275), the need for finding new molecules of this type may be limited. However, our assay is also sensitive to small molecules that inhibit deoxyHbS oligomerization independent of oxygen affinity, and we have found several such compounds here although their mechanism of action remains unknown. There is little structural similarity among gabazine, chlormezanone, and voxelotor. This is consistent with chlormezanone and gabazine not shifting oxygen binding affinity and suggests that they are non-covalent binders. Chlormezanone and gabazine have unique core structures from each other and, most likely, a unique binding mode to HbS. We can only speculate about their mechanism of action, but they might act via two possible mechanisms: 1) in the presence of hit compounds, hemoglobin might adopt a slightly different conformation where the acceptor hydrophobic pocket becomes inaccessible to 06 valine to oligomerize; and 2) hit compounds might be competing with 06 valine for interaction with the acceptor hydrophobic pocket on the surface of an adjacent Hb molecule. Regardless of the mechanism, the low potency of chlormezanone and gabazine in the fibrillization assay suggest that they are likely not strong lead molecules. Additionally, these compounds have known adverse effects (chlormezanone (Roupe et al., 1986, Int Arch Allergy Appl Immunol 80, 145-151)) and off-target effects (gabazine (Behrens et al., 2007, Eur J Neurosci 25, 2170-2181; Iqbal et al., 2011, Bioorganic & Medicinal Chemistry Letters 21, 4252-4254)). In fact, it would be highly unusual to find a high-potency lead in such a small library, and thus this initial study was intended as proof-of-principle for this new approach. To realize the promise of this novel mechanism as a clinically effective approach will depend on the existence of a high-specificity binding site for this class of compounds, which we have not proven here. The hit rate in our pilot screen offers hope that such compounds could be found by screening large libraries and using medicinal chemistry.

The development of this HTS platform comes at a critical time in the development of new therapies for SCD. The World Health Organization and United Nations recognize SCD as a global health issue (Mburu et al., 2019, Int J Lab Hematol 41 Suppl 1, 82-88). According to the Center for Disease Control, SCD affects over 100,000 individuals in the US, primarily Black communities, worsening the health disparity among minority populations (Lee et al., 2019, Public Health Rep 134, 599-607). Furthermore, in resource-poor nations more than 90% of children with SCD do not survive to adulthood (Uyoga et al., 2019, Lancet Glob Health 7, e1458-e1466; Odame, 2014, Nature 515, S10). Hence, there is a deep need to accelerate therapeutic discovery. There is also a growing recognition that gene therapy, while potentially curative, is too costly and complex to become a broadly applicable strategy for the millions with SCD worldwide. Thus, the tools for developing new small molecule therapies must be expanded and enhanced. It is worth noting that of the currently approved therapies, only voxelotor would have been discovered using our screening approach. While HbS polymerization is the core molecular mechanism of the disease, there is also room for assays to discover therapies that target other elements of the pathophysiology. In the context of inhibiting polymerization, we know of at least two other efforts to screen drugs for SCD, including recent work by Pfizer that resulted in a drug now in clinical trial (Gopalsamy et al., 2021, J Med Chem 64, 326-342), and work by Bill Eaton and colleagues at NIH that has found promising hits using a cell-based HTS assay (Dunkelberger et al., 2018, J Phys Chem B 122, 11579-11590; Li et al., 2017, Proceedings of the National Academy of Sciences of the United States of America 114, E689-E696). After seven decades, traditional large-scale drug discovery campaigns for SCD are now underway and promise to revolutionize treatment options for this terrible disease.

### Materials and Methods

Preparation and characterization of non-fibrillar oligomers: Oligomerization of deoxy-HbS at sub-nucleating concentrations was measured using dynamic light scattering (DLS). Brookhaven's 90 plus particle size analyzer equipped with a 633 nm He-Ne laser was used to measure the effective diameter of HbS. This laser produces a vertically polarized beam with a wavelength of 632.8 nm. Effective diameter was measured at a scattering angle of 90°. For this assay, HbS (2 mg/ml) was dissolved in filtered PBS and then centrifuged at 13000 g for 5-10 minutes at 4° C. to remove the debris. After centrifugation, HbS supernatant was recovered. To make dust-free samples for DLS experiments, all samples were passed through a 0.2 mm filter. Protein concentration was measured spectrophotometrically using Beer's law. Next, HbS (25 mM) sample was incubated with hit compounds at room temperature for two hours. Next, HbS was vacuumed degassed for 5-10 minutes. Then, oligomerization of deoxy-HbS was measured using DLS. Stability of non-fibrillar oligomers was determined by taking the DLS readings every minute at 25° C. for two hours. All DLS data reported here are taken with unlabeled protein.

Development of HTS platform for identification of small molecules that inhibit in vitro HbS nucleation: Sickle hemoglobin (HbS) (sigma) was labeled with either fluorescent donor (rhodamine red) or acceptor (Cy5) dyes (Thermo fisher scientific) via a cysteine thiol group. For labeling, lyophilized HbS (10 mg/ml) was dissolved in PBS (Sigma) and then centrifuged at 13000 g for five minutes at 4° C. to remove the debris. After centrifugation protein sample was incubated with TCEP (0.5-1 mM) for 20 minutes at room temperature to reduce the cysteine side-chain. This protein mixture was incubated with 1.5 mM dye (dissolved in DMSO or water) for two hours in the dark at room temperature or overnight at 4° C. Unreacted free dye was removed by dialysis against PBS. The degree of labeling was calculated by comparing the absorption of the fluorophore conjugated hemoglobin at 280 nm to the absorbance of the dye at its maximum wavelength. The dye concentration was measured by absorbance, using extinction coefficients of 250,000 M −1 cm -1 for Cy5 conjugates and 88000 M −1 cm -1 for Rhodamine Red conjugates.

High-Throughput Screening of LOPAC Library to Identify Compounds That Inhibit HbS Oligomerization Under Deoxy Condition: The LOPAC compounds (Thermo Fisher Scientific) were received in 96-well plates. Assay plates were prepared by transferring 5 nL of the 10 mM compound stocks or DMSO from the source plates to 1536-well black polypropylene plates (Greiner), using an Echo 550 acoustic dispenser (Labcyte). During screening, donor sample (HbS/Rhodamine Red) was mixed with acceptor (HbS/Cy5) at 1:5 ratios. Then protein samples were dispensed (5 μL, 25 μM) by a Multidrop Combi Reagent Dispenser (Thermo Fisher Scientific) into the 1536-well assay plates containing the compounds and incubated at room temperature for two hours. After incubation, protein samples were degassed for 10-15 minutes using vacuum argon degassing method. Lifetime measurements were taken by the fluorescence lifetime plate reader (Fluorescence Innovations, Inc).

Fluorescence Data Acquisition: We tested the functionality of the HbS FRET pair by measuring lifetimes of oxy-and deoxy HbA and HbS using a fluorescence lifetime plate reader (FIG. 3A). Lifetimes are reciprocally related to FRET, so a decrease in lifetime corresponds to an increase in FRET. Donor fluorescence was excited with a 532 nm laser (Teem Photonics SNG-20F-100, Meylan, France), delivering pulses of -0.3 μJ energy at a 20 kHz repetition rate, and emission was filtered with 546 nm long pass and 586/20 nm band pass filters. This filter was selected to exclude acceptor fluorescence, as only the lifetime of the donor fluorescent protein is required to calculate FRET (Equation (1)). The photomultiplier tube (PMT) voltage was adjusted so that the peak signals of the instrument response function (IRF) and FRET biosensor were similar.

High-Throughput Screening Data Analysis: Time resolve-fluorescence waveforms for each well were fitted with single-exponential decays using least-squares minimization global analysis software (Petersen et al., F2014, Rev Sci Instrum 85, 113101). FRET efficiency (E) was determined as the fractional decrease of donor lifetime (T_D ), due to the presence of acceptor fluorophore (T_DA) using equation 1:

$$E = 1 - ( \frac{{τ}_{D A}}{{τ}_{D}} ) \qquad ( 1 )$$

Assay quality was determined based on controls (phenylalanine as a positive control and DMSO as negative control) on each plate, as indicated by the Z∧′ factor (Zhang et al., 1999, J Biomol Screen 4, 67-73):

$${Z}^{′} = 1 - \frac{3 ( {σ}_{p} + {σ}_{n} )}{\vert {μ}_{p} - {μ}_{n} \vert} \qquad ( 2 )$$

where σₚ and σₙ are the standard deviations (SDs) and μₚ and μₙ are the means of the positive and negative controls, respectively.

FRET Dose-Response Assay: The hit compounds, chlormezanone, gabazine and phosphoramidon disodium, were purchased from Sigma. These drug compounds were dissolved in DMSO to make 20 mM stock solution. Hits were screened at seven different concentrations. Protein samples were dispensed into the 96-well assay plates and incubated with hit compounds at room temperature for two hours. After incubation, protein samples were degassed for 10 minutes using vacuum argon degassing method. Lifetime measurements were taken by the fluorescence lifetime plate reader (Fluorescence Innovations, Inc).

Polymerization of deoxy-HbS in High Phosphate Buffer: Effect of hit compounds on polymerization of deoxy-HbS in high potassium phosphate buffer was monitored using spectramax i3X. High concentration phosphate buffer (1.895 M potassium phosphate) was made by dissolving solid monobasic and dibasic potassium phosphate in deionized water with final pH 7.3. Purified HbS was concentrated in high phosphate buffer by buffer exchange using spin concentrators at 4° C. On the day of experiment, temperature of spectramax i3X was set to 37° C. Next, plastic 1 mL UV cuvettes were placed on ice and 950 uL of 1% MBS in 1.895 KPi were quickly pipetted into each cuvette. Then, 50 ml of HbS (24 mg/ml, final concentration 1.2 mg/ml) were added to the cuvette and mix, and placed in spectramax. The cuvette reached temperature in about 3 minutes and the HbS was completely converted into deoxy-HbS in this time. The delay time for 1.2 mg/mL HbS was around 470 seconds and the midpoint was around 520 seconds. Next, a 700 nm absorbance reading was taken every 3 seconds. Growth curves were normalized by setting the initial linear part of the curve typically from 500-1500 seconds to baseline at zero and then normalizing the end of the curves (which would be completely fibrillized) to 100% fibrillized. Lines were fit to the linear part of the growth curves, and the slopes were taken as the growth rate and compared between conditions.

Blood Sample Collection and Preparation for Microfluidic Assays: Sickle cell patient blood samples were collected under protocols approved by the Institutional Review Boards at Children's Minnesota Hospital, the University of Minnesota Medical Center, and the University of Minnesota. Patient blood samples were collected into sodium citrate vacutainer tubes and stored at 4° C. The samples were prepared for the microfluidic experiments by washing twice with Dulbecco's phosphate buffered saline (DPBS) after centrifugation for 5 minutes at 400g. Packed red blood cells were then isolated by centrifuging the washed samples for 10 minutes at 400g and removing all remaining DPBS. The packed red blood cells were added to fresh DPBS to achieve a target hematocrit of 25%. Each compound of interest was added to the sample to reach a target concentration of 500 uM and incubated at 37° C. for 1 hour before being run on the microfluidic device. The compounds were dissolved in DMSO at a concentration of 100 mM and stored at −20° C. or −80° C. prior to use.

Microfluidic Setup and Experimental Protocol: A previously described (Valdez et al., 2019, APL Bioengineering 3, 046101; Geisness et al., 2022, Haematologica, 107(6); Hansen et al., 2020, Br J Haematol, (2019). Br J Haematol. 188(6): 985-993), polydimethylsiloxane (PDMS) microfluidic device replicating the geometry of the post-capillary venules was used to study blood rheological behavior. FIG. 7 shows the device layout with the inputs and outputs for each of the three layers. The microfluidic channels were primed by flowing a 2% bovine serum albumin (BSA) in DPBS solution through the device to passivate the PDMS surface. Blood was then added to the inlet port and flowed through the device under a constant driving pressure to achieve an initial velocity of 700 mm/s (PCD-15PSIG, Alicat Scientific). At the bifurcation, blood is diverted to two 15 mm square channels—a bypass channel and an experimental channel. Separate gas inlets supply oxygen to each of the two blood channels independently via diffusion from the overlaid gas layer. The bypass channel is supplied with air and maintained under normoxic conditions. In the experimental channel, a gas mixing system is used to control the oxygen level on the device and recorded with a fiber optic oxygen sensor ((NeoFox-GT, Ocean Optics, Dunedin, Fla.). Air (21% 02) is supplied to the experimental region of the device for 3 minutes followed by 5 minutes of hypoxia. The degree of hypoxia is increased with each successive cycle in 2% 02 increments (FIG. 9A). A high-speed camera continuously collects four-frame bursts of images of the channel at 40× magnification on a Zeiss Axio Vert.A1 microscope (Carl Zeiss, Oberkochen, Germany). The bursts of images are analyzed frame-by-frame in real time using the Kanade-Lucas-Tomasi (KLT) feature tracking algorithm in Matlab (Mathworks, Natick, Mass.) to determine the average velocity of the blood through the channel (FIG. 8A).

Microfluidic Measurement of RBC Oxygen Saturation: The Quantitative Absorption Cytometry assay described previously (Di Caprio et al., 2019, Proc Natl Acad Sci USA 116, 25236-25242; Di Caprio et al., 2015, Proc Natl Acad Sci USA 112, 9984-9989), takes images of single red blood cells (RBC) under alternating 410 nm and 430 nm LEDs. Due to the unique absorption spectra of oxy and deoxy hemoglobin the transmitted light at 410 nm and 430 nm permits measurement of the single cell saturation can be calculated. The cells are perfused through a microfluidic device with oxygen control that permits measurement of the oxygen saturation of a population of RBC under defined oxygen tension. The polydimethysiloxane (PDMS) microfluidic device for this assay comprises two layers- a gas layer and a blood layer bonded to a glass coverslip. These two layers are separated by 100 μm of PDMS. The gas layer allows for precise control of oxygen tension that will diffuse to the blood layer; the blood layer contains a 30 mm diffusion section to allow red blood cells to reach steady state saturation and a 5 mm observation section to imaging RBCs.

To prepare RBC for measurement, blood samples were washed and resuspended in 10% w/v BSA in PBS, and 0.5 μL of 100 mM therapeutic in DMSO was added to the sample. The sample was then incubated on a shaker plate at 37° C. for one hour. After incubation samples were washed again and 10 μL packed red blood cells was added to a solution of 288 μL 25% Human Serum Albumin (HAS) solution (Gemini Bio).

In this assay, we prepared samples identically to the preparation for blood flow by incubating with drug at 25% hematocrit, and then we diluted the samples to 1% hematocrit with 500 mM drug in the dilution buffer. The use of drug in the running buffer after dilution of the RBC was done to ensure that we did not dilute any drug effects relative to the whole blood studies. This preparation resulted in higher modification (˜100%) of hemoglobin with voxelotor than in the whole blood studies, yielding a large shift in P50 (FIG. 10A).

The complete disclosure of all patents, patent applications, and publications, and electronically available material (including, for instance, nucleotide sequence submissions in, e.g., GenBank and RefSeq, and amino acid sequence submissions in, e.g., SwissProt, PLR, PRF, PDB, and translations from annotated coding regions in GenBank and RefSeq) cited herein are incorporated by reference in their entirety. Supplementary materials referenced in publications (such as supplementary tables, supplementary figures, supplementary materials and methods, and/or supplementary experimental data) are likewise incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

What is claimed is:

1. A method for identifying a compound that alters intermolecular contact between two proteins, the method comprising:
providing a composition comprising low molecular weight sickle hemoglobin (HbS) assemblies;
adding a test compound to the composition; and
measuring a change in the structure of the low molecular weight HbS assemblies.

2. The method of claim 1, wherein the low molecular weight HbS assemblies comprise a first sickle hemoglobin (HbS) tetramer and a second HbS tetramer,
wherein the first HbS tetramer comprises a donor chromophore,
wherein the second HbS tetramer comprises an acceptor chromophore,
wherein the first and second HbS tetramers are deoxygenated,
wherein the first and second HbS tetramers form a low molecular weight HbS assembly, and
wherein the measuring comprises measuring fluorescence resonance energy transfer (FRET) of the donor chromophore, the acceptor chromophore, or the combination thereof.

3. The method of claim 2, wherein a difference between the FRET in the presence of the test compound and the FRET in the absence of the test compound is not detected.

4. The method of claim 2, wherein a difference between the FRET in the presence of the test compound and the FRET in the absence of the test compound is detected.

5. The method of claim 1, wherein the difference indicates that the test compound alters the FRET of the donor chromophore, the acceptor chromophore, or the combination thereof.

6. The method of claim 1, wherein the ratio of donor to acceptor is 7:1, 6:1, 5:1, 4:1, 3:1: 1:1, 1:2, 1:3, 1:4, 1:5, 1:6, or 1:7.

7. The method of claim 1, wherein the providing comprises incubation of the composition at a concentration of 25 μM in the presence of an oxygen scavenger.

8. The method of claim 2, wherein the FRET of the donor probe is changed in the presence of the test compound.

9. The method of claim 8, wherein the FRET of the donor probe is reduced in the presence of the test compound.

10. The method of claim 2, wherein the FRET of the donor probe is unchanged in the presence of the test compound.

11. The method of claim 1, wherein the measuring comprises high-throughput screening.

12. The method of claim 1, wherein the measuring comprises measuring dynamic light scattering (DLS) of the low molecular weight HbS assemblies.

13. The method of claim 12, wherein a difference between the DLS in the presence of the test compound and the DLS in the absence of the test compound is not detected.

14. The method of claim 12, wherein a difference between the DLS in the presence of the test compound and the DLS in the absence of the test compound is detected.

15. The method of claim 14, wherein the difference indicates that the test compound alters the effective diameter of the low molecular weight HbS assemblies.

16. A low molecular weight HbS assembly comprising at least 2 HbS monomers, wherein the HbS monomer comprises a tetramer, the tetramer comprising two alpha subunits and two beta subunits, wherein the beta subunits each comprise the sickle cell point mutation.

17. The low molecular weight HbS assembly of claim 16, wherein one or both beta subunits comprise a chromophore.

18. The low molecular weight HbS assembly of claim 16, wherein the stable non-fibrillar oligomer comprises two populations of HbS monomers, wherein the first population comprises a donor chromophore and the second population comprises an acceptor chromophore.

18. A composition comprising the low molecular weight HbS assembly of claim 16.

20. The composition of claim 19, wherein the concentration of HbS monomers is at least 15 μM and no greater than 1 mM.
